(19) United States
(12) Patent Application Publication
(10) Pub. No.: US 2010/0185699 A1
(43) Pub. Date: Jul. 22, 2010

(54) SYSTEM AND METHOD FOR REPRESENTING N-LINKED GLYCAN STRUCTURES

(75) Inventors: DONG YUP LEE, SINGAPORE (SG); FARAAZ YUSUFI, SINGAPORE (SG)

Correspondence Address: KNOBBE MARTENS OLSON & BEAR LLP, 2040 MAIN STREET, FOURTEENTH FLOOR, IRVINE, CA, 92614, US

(73) Assignee: AGENCY FOR SCIENCE, TECHNOLOGY AND RESEARCH, SINGAPORE (SG)

(21) Appl. No.: 12/664,883

(22) PCT Filed: Jun. 13, 2008
(86) PCT No.: PCT/SG08/00212

§ 371 Date: Dec. 15, 2009

Related U.S. Application Data
(60) Provisional application No. 60/929,163, filed on Jun. 15, 2007.

(51) Int. Cl. G06F 17/00; C07H 3/06; G06F 7/00

(52) U.S. Cl. 707/803; 536/123.1; 707/E17.004; 707/E17.044

(57) ABSTRACT

A fixed-length alpha-numeric code for representing N-linked glycan structures commonly found in secreted glycoproteins from mammalian cell cultures. The code employs a pre-assigned alpha-numeric index to represent the monosaccharides attached in different branches to the core glycan structure. The present branch-centric representation allows visualization of the structure while the numerical nature of the code makes it machine readable. A difference operator can be defined to quantitatively differentiate between glycan structures for further analysis. The code can be incorporated in a retrievable format into an information management system. A method is also provided for representing the structure of at least a portion of an oligosaccharide, using the fixed-length alpha-numeric code.

### CROSS-REFERENCE TO RELATED APPLICATIONS

The present patent application is based on, and claims priority from, U.S. provisional Application No. 60/929,163, filed Jun. 15, 2007, which is incorporated herein by reference in its entirety.

### BACKGROUND OF THE INVENTION

1. Field of the Invention

The present invention relates to a system for describing glycan structures that can be easily stored and interpreted by computers.

2. Related Art

Glycans are complex chains of oligosaccharides that play critical roles in several structural and modulatory functions in cells. Although glycans are considered as one of the most important classes of molecules after DNA and proteins, the development of informatics methods to support and advance their research has lagged behind those available for other types of data. It is only in recent years that there has been an increase in the availability of informatics resources such as glycan databases and algorithms for analyzing glycan structures and their interactions (Pérez S, Mulloy B (2005) “Prospects for glycoinformatics.” Curr Opin Struct Biol 15:517-524 “(“Pérez et al.”). Such disparity is mainly attributable to the structural complexity of carbohydrates compared to the simpler linear structure of DNA and proteins. While nucleotide and amino acid residues can be represented by four and twenty letters respectively, glycan sequences are comprised of a larger number of base residues and contain additional information on linkages and branching (von der Lieth C W (2004) “An endorsement to create open databases for analytical data of complex carbohydrates.” J Carbohydr Chem 23:277-297 (“von der Lieth I”); Laine R A (1994) “A calculation of all possible oligosaccharide isomers both branched and linear yields 1.05×10(12) structures for a reducing hexasaccharide: the Isomer Barrier to development of single-method saccharide sequencing or synthesis systems.” Glycobiology 6:759-767). As a result, several research projects suffer from the lack of a suitable digital format that would render glycan data freely available to other researchers and interoperable in different applications (von der Lieth C W, Bohne-Lang A, Lohmann K K, Frank M (2004) “Bioinformatics for glycomics: status, methods, requirements and perspectives.” Brief Bioinform 5:164-178). Thus, it is necessary to develop a simple, flexible and versatile data format for the representation of glycan structures that is easily understood by scientists and also readable by computers (Brazma A, Krestyaninova M, Sarkans U (2006) “Standards for systems biology.” Nat Rev Genet 7:593-605.

Currently, there are a few nomenclatures available to describe glycan structures, some of which are illustrated in FIGS. 1a-1d. The IUPAC-IUBMB (International Union for Pure and Applied Chemistry and International Union for Biochemistry and Molecular Biology) provides extended and abbreviated text formats to fully describe glycan structures (McNaught A D (1997) “Nomenclature of carbohydrates” (recommendations 1996). Adv Carbohydr Chem Biochem 52:43-177). The abbreviated three-letter codes stand for individual monosaccharide units, with each unit accompanied by an anomeric descriptor, as well as stereochemistry and linkage information. The IUPAC descriptions are, however, ambiguous and not sufficient to comprehensively describe all glycans in a computer readable format. To overcome this limitation, LINUCS (LInear Notation for Unique description of Carbohydrate Sequences) was developed to create a linear representation of the glycan by extending IUPAC description along with the glycosidic linkage information (Bohne-Lang A, Lang E, Forster T, von der Lieth CW (2001) “LINUCS: linear notation for unique description of carbohydrate sequences.” Carbohydr Res 336:1-11). Another available format is Glycominds' Linear Code™ which exploits a special lookup table for determining the order of branching (Banin E, Neuberger Y, Altshuler Y, Halevi A, Inbar O, Nir D, Dukler A (2002) “A novel linear code nomenclature for complex carbohydrates.” Trends Glycosci Glycotechnol 14:127-137). The monosaccharide units and linkages are represented by one- to two-letters in this representation. Recently, the growing popularity of XML as a data descriptive language has also led to the proposal of XML-based representations of glycan structures such as GLYDE (Sahoo S S, Thomas C, Sheth A, Henson C, York W S (2005) “GLYDE—an expressive XML standard for the representation of glycan structure.” Carbohydr Res 340:2802-2807) and CabosML (Kikuchi N, Kameyama A, Nakaya S, Ito H, Sato T, Shikanai T, Takahashi Y, Narimatsu H (2005) “The carbohydrate sequence markup language (CabosML): an XML description of carbohydrate structures.” Bioinformatics 21:1717-1718). There are additional formats available for describing glycan structures which have been reviewed elsewhere (Pérez et al; von der Leith I; Toukach P, Joshi H J, Ranzinger R, Knirel Y, von der Lieth C W (2007) “Sharing of worldwide distributed carbohydrate-related digital resources: online connection of the bacterial carbohydrate structure database and GLYCOSCIENCES.de.” Nucleic Acids Res 35:D280-286).

Mammalian cell lines are ideal for producing recombinant proteins that require post-translational modifications such as glycosylation. Since glycosylation has an effect on various biological properties such as folding, stability and efficacy, the quality of secreted proteins is dependent on the consistency of attached glycan structures. Thus, studying the complex glycosylation reaction pathway in an effort to control the diversity of protein glycosylation is a very active area of research.

It is to the solution of these and other problems that the present invention is directed.

### SUMMARY OF THE INVENTION

It is accordingly a primary object of the present invention to provide a compact notation for describing glycan structures that can be easily stored and interpreted by computers.

It is another object of the present invention to provide a simplified alpha-numeric representation of glycan structures that can facilitate the development of computer aided analysis tools to study these complex pathways.

It is still another object of the present invention to provide a simplified alpha-numeric representation of glycan structures that can replace text based representations.

It is still another object of the invention to provide a method for representing the structure of at least a portion of an oligosaccharide.

These and other objects of the present invention are achieved by an alpha-numeric code, hereinafter referred to as the “GlycoDigit code,” for the description of N-linked glycan structures that are commonly observed in secreted glycoproteins from engineered mammalian cell lines such as Chinese hamster ovary (CHO) cells.

In one aspect of the invention, a six character alpha-numeric code is used to describe glycan structures on the basis of the monosaccharide chains attached to the different branches of the core structure. In another aspect of the invention, structures in the GlycoDigit code are represented by seven digit-letter pairs for an overall fixed length of fourteen characters. The numeric component of the alpha-numeric code allows for the development of a difference operator and an algorithm to make convenient comparison of glycans based on the unique alpha-numeric code for each structure.

Other objects, features and advantages of the present invention will be apparent to those skilled in the art upon a reading of this specification including the accompanying drawings.

### BRIEF DESCRIPTION OF THE DRAWINGS

The invention is better understood by reading the following Detailed Description of the Preferred Embodiments with reference to the accompanying drawing figures, in which like reference numerals refer to like elements throughout, and in which:

FIG. 1a is a symbolic representation of N-linked glycan structures using symbols adopted from the nomenclature proposed by the Oxford Glycobiology Institute (UK) to represent a structure pictorially.

FIG. 1b is a full-word representation of the N-linked glycan structures of FIG. 1A.

FIG. 1c is a representation of the N-linked glycan structures of FIG. 1A, using the LINUCS format.

FIG. 1d is a representation of the N-linked glycan structures of FIG. 1A, using the Linear Code™.

FIG. 2 depicts the pentasaccharide core structure common to all N-linked glycans sharing a common pentasaccharide core structure, along with possible sites where additional branches of sugars can attach.

FIG. 3 shows the possible branching from the core structure of FIG. 2, and the corresponding position of each digit for the antennary for a six character alpha-numeric code in accordance with a first embodiment of the GlycoDigit code of the present invention.

FIG. 4a is a pictorial representation of a complex N-linked glycan and its corresponding representation using the first embodiment of the GlycoDigit code in accordance with the present invention.

FIG. 4b is a pictorial representation of a high-mannose N-linked glycan and its corresponding representation using the first embodiment of the GlycoDigit code in accordance with the present invention.

FIG. 4c is a pictorial representation of a hybrid N-linked glycan and its corresponding representation using the first embodiment of the GlycoDigit code in accordance with the present invention.

FIG. 5a is a pictorial representation of a complex N-linked glycan and its corresponding representation using a second embodiment of the GlycoDigit code in accordance with the present invention.

FIG. 5b is a pictorial representation of a high-mannose N-linked glycan and its corresponding representation using the second embodiment of the GlycoDigit code in accordance with the present invention.

FIG. 5c is a pictorial representation of a hybrid N-linked glycan and its corresponding representation using the second embodiment of the GlycoDigit code in accordance with the present invention.

FIGS. 6a-6f illustrates a step-by-step representation of the corresponding GlycoDigit code for the complex type structure represented in FIG. 6a, using the second embodiment of the GlycoDigit code in accordance with the present invention.

FIG. 7 illustrates using a difference operator to find the structural differences between two glycans, using their corresponding GlycoDigit codes in accordance with the first embodiment of the present invention.

FIG. 8 illustrates using a difference operator to find the structural differences between a complex glycan structure and a hybrid N-linked glycan structure, using their corresponding GlycoDigit codes in accordance with the second embodiment of the present invention.

FIG. 9 shows two glycans and the reaction steps needed to convert one structure to another, using the first embodiment of the GlycoDigit code in accordance with the present invention.

FIG. 10 shows the pseudocode for the isrxn and rxm_matrix functions used to populate an adjacency matrix of glycan reactions.

FIG. 11a is a visualization of a network of glycans and reaction links for a reduced data set of 64 two-branched glycans, arranged in a hierarchical way.

FIG. 11b is an enlargement of the area designated 11b in FIG. 11a.

FIG. 12a is a visualization of the entire glycosylation network for 1,024 complex type glycans commonly secreted in CHO cells, arranged in a hierarchical way.

FIG. 12b is an enlargement of the area designated 12b in FIG. 12a.

FIG. 12c is an enlargement of the area designated 12c in FIG. 12b.

FIG. 13 is a key for the symbols used in FIGS. 1a, 2, 3, 4a-4c, 5a-5f, 6a-6f, 7, 8, and 9.

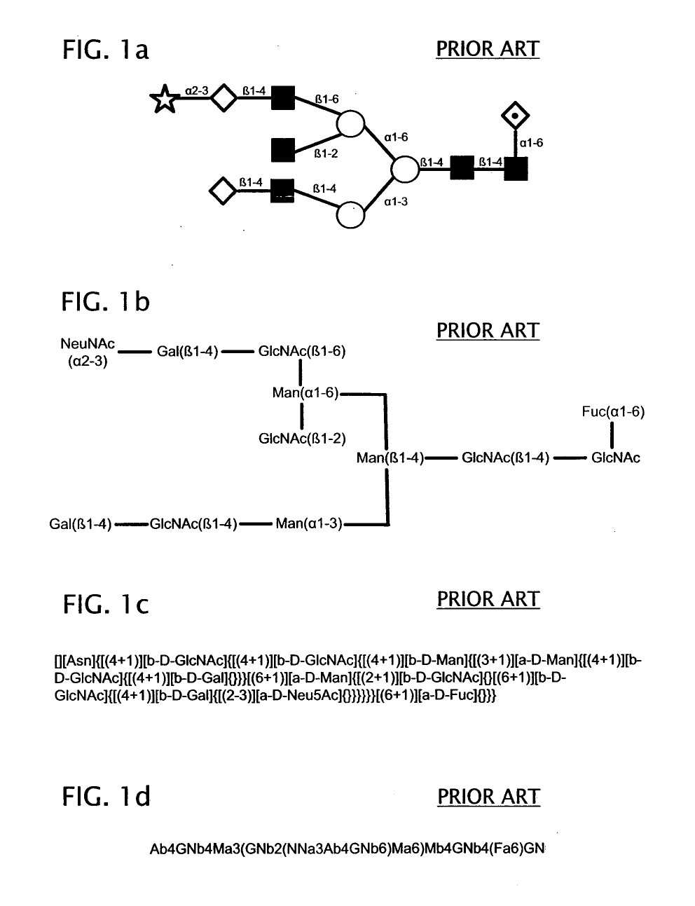

### DETAILED DESCRIPTION OF THE PREFERRED EMBODIMENTS

In describing preferred embodiments of the present invention illustrated in the drawings, specific terminology is employed for the sake of clarity. However, the invention is not intended to be limited to the specific terminology so selected, and it is to be understood that each specific element includes all technical equivalents that operate in a similar manner to accomplish a similar purpose.

Methods

One aspect of the invention is a method for representing the structure of at least a portion of an oligosaccharide. Preferably, the representation will be one which is easily stored on and analyzed by a computer. The method of the invention as described below may be applied to produce the specific “GlycoDigit” code described herein, but it will be understood that it may also applied to generate different representations of the structure of an oligosaccharide.

The first part of the method of the invention involves the creation of the representational system, and comprises the following steps:
(a) selecting a base oligosaccharide structure;
(b) identifying a number of possible substitution points on the base structure selected in step (a) and assigning a position to each one;
(c) assigning a two-character code to a substitution point from step (b), where “character” means any unique identifier, the two-character code having a first character and a second character;
(d) assigning one or more unique identifiers for the first character of the two-character code and one or more unique identifiers for the second character of the two-character so that the first character and the second character together uniquely identify a residue on a specific substitution point identified in step (b); and
(e) repeating step (d) for each substitution point so that each substitution point identified in step (b) has a set of two-character codes which identify the possible residues for that substitution point.

In step (a), a base oligosaccharide structure is selected. Preferably, this base structure will be one which is present in a great many of the oligosaccharide structures of interest. The “larger” the base structure (i.e. the greater the number of common structural features in the oligosaccharides of interest) the less complicated the representational system need be.

In step (b), each of the possible substitution points on the base structure are identified. Typically, each possible substitution point is assigned a number, from 1 to x, which will correspond to a position in the final structural representation. The larger the number of substitution points, the more complicated a structure the method can represent. In step (c), a two-character code is selected, where “character” means any unique identifier. Typically, one character will be a number and one will be a letter, but both could be numbers, or letters. Non-roman alphabets can also be used, e.g. Russian, Greek, Hebrew, etc.

In step (d), meanings for the characters selected in step (c) are assigned. An example of this is discussed in detail below with respect to the GlycoDigit code, but any system may be used. The combination of meanings for each two-character grouping is used to specifically define the residue present at each preselected substitution point. It is important to note that it is not necessary that the identifiers be able to identify every single possible residue at a particular substitution point, so long as all the ones of interest are covered. In step (e), step (d) is repeated for each of the substitution points identified in step (b).

The second part of the claimed method involves applying the system developed above to a particular oligosaccharide:
(f) reviewing the structure of an oligosaccharide structure containing the base oligosaccharide structure selected in step (a) and optionally one or more residues on that base structure; and
(g) assigning the two-character codes to the residues on the oligosaccharide structure of step (f) to match the two-character codes developed in steps (d) and (e) and recording them in the positions assigned in step (b).

It will be apparent to those of skill in the art that the GlycoDigit codes described in detail hereinafter can be applied using this method.

N-Linked Glycan Structures

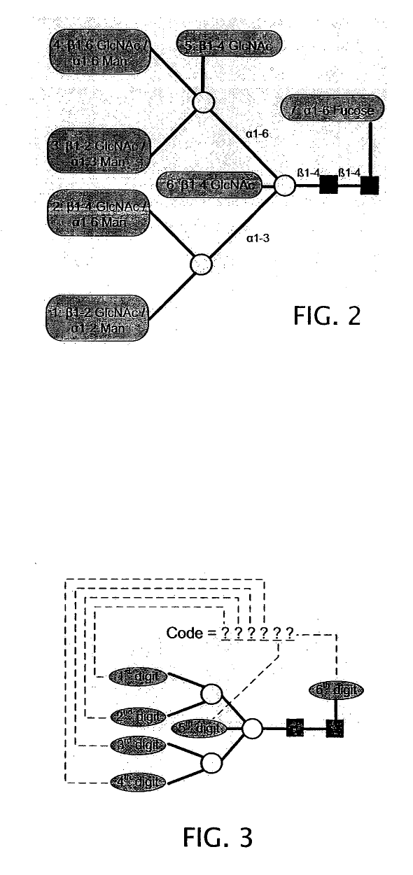

N-linked glycosylation occurs in all eukaryotic cells with N-linked glycans sharing a common pentasaccharide core structure depicted in FIG. 2. Several monosaccharide chains can attach to this core structure at different linkage positions by the action of different glycosyltransferase enzymes. N-linked glycan structures can be of the high-mannose, complex, or hybrid subtype. High-mannose N-linked glycans contain only mannose (Man) residues linked to the core structure, while complex N-linked glycans have N-acetylglucosamine (GlcNAc) residues attached to the core. The hybrid subtype contains branches with both GlcNAc and unsubstituted mannose residues (Varki A et al. (eds) (1999) Essentials of glycobiology. New York (USA): Cold Spring Harbor Laboratory Press (“Varki et al”).

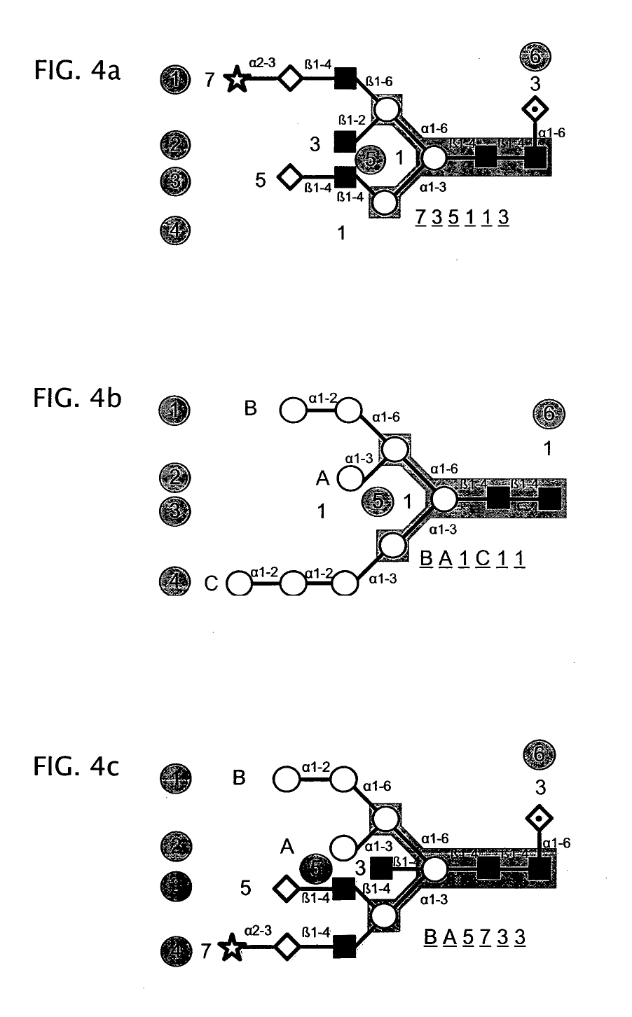

In a first embodiment of the invention, shown in FIGS. 4a-4c, a six character alpha-numeric code is used to describe glycan structures on the basis of the monosaccharide chains attached to the different branches of the core structure shown in FIG. 2. The first four characters correspond to the four possible antennaries linked to the upper and lower core mannose residues, while the fifth and sixth characters represent a bisecting GlcNAc and a fucose group respectively. FIG. 3 shows the possible branching from the core structure and also, the corresponding position of each character for the antennary.

The first four branches are represented by odd numbers if the branch is a complex type while high-mannose branches are represented by letters. Complex branches terminating as a GlcNAc, galactose or neuraminic acid residue are represented by the number 3, 5 or 7 respectively. The mannose residues of hybrid and high-mannose N-linked glycans are represented by the letters A-F, with each letter designated as an even number, i.e., A=2, B=4, C=6 etc. For each branch, the letter value corresponds to double the number of mannose residues attached to that branch, i.e. A=2 implies that one mannose residue is attached, B=4 implies that two mannose residues are attached, etc. The fifth and sixth characters have a value of 3 if a bisecting GlcNAc and fucose residue are present respectively. If a branch is not present, its corresponding digit is 1. Further rules are defined that limit the number of mannose residues that can be attached to a structure and which combination of complex and high mannose branches are allowed. From these definitions, the GlycoDigit code can be used to describe the structures of 5100 glycans.

Glycosyltransferases are enzymes that sequentially add one monosaccharide at a time to glycan structures. Six GlcNAc transferases (GlcNAcT I-VI) can add GlcNAc to the three core mannose in different linkages. As shown in FIG. 2, on the α1-3 linked core mannose, GlcNAcT I and IV add residues in the β1-2 and β1-4 linkages, respectively. Similarly, on the α1-6 mannose GlcNAcT II, V and VI attach β1-2, β1-6 and β1-4 linked residues. Additionally, one bisecting GlcNAc can attach through a β1-4 link to the central core mannose (Campbell C, Stanley P (1984) “A dominant mutation to ricin resistance in Chinese hamster ovary cells induces UDP-GlcNAc: glycopeptide beta-4-N-acetylglucosaminyltransferase III activity.” J Biol Chem 259:13370-13378; Sburlati A R, Umana P, Prati E G, Bailey J E (1998) “Synthesis of bisected glycoforms of recombinant IFN-beta by over-expression of beta-1,4-N-acetylglucosaminyltransferase III in Chinese hamster ovary cells.” Biotechnol Prog 14:189-192 (“Sburlati et al”); Umana P, Jean-Mairet J, Moudry R, Amstutz H, Bailey J E (1999) “Engineered glycoforms of an antineuroblastoma IgG1 with optimized antibody-dependent cellular cytotoxic activity.” Nat Biotechnol 17:176-180 (“Umana et al”)). Finally, a fucose residue can attach in α1-6 linkage to the core GlcNAc that connects to the asparagine amino-acid on the protein (Varki et al).

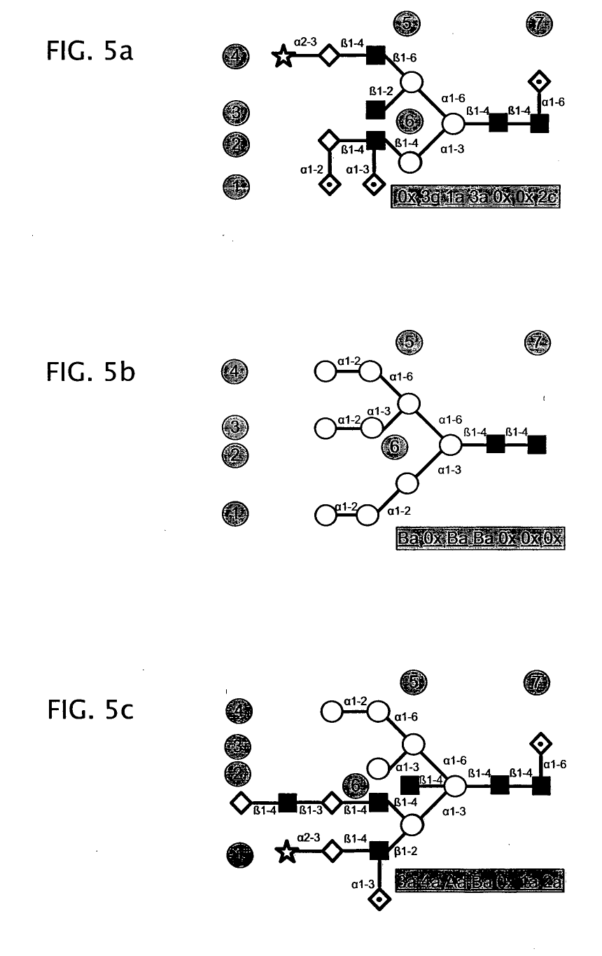

Based on these seven possible linkage sites, in a second embodiment of the invention, shown in FIGS. 5a-5c, the GlycoDigit code uses seven digit-letter pairs to represent glycan structures. Each digit-letter pair in the second embodiment of the GlycoDigit code corresponds to a branch connected from the core structure illustrated in FIG. 2. The first six digit-letter pairs correspond to the six possible branches linked to the upper and lower core mannose residue. A bisecting GlcNAc between the mannoses is represented by the sixth digit-letter pair, and the final seventh position corresponds to fucose molecules that can be attached to the core or peripheral GlcNAc residues. The digit portion of each pair corresponds to the number of monosaccharides attached at that branch while the letter serves as an index to a table containing additional information about the type of linkage and the specific sugar molecule added.

Table 1 lists which linkage each digit-letter pair corresponds to in the second embodiment of the GlycoDigit code. High mannose and hybrid structures can be represented by using the first four digit-letter pairs to correspond to α1-2, α1-3 and α1-6 linked mannose chains attached to each of the two mannose residues in the core structure as shown in FIG. 2. In order to differentiate between complex and high mannose branches, the number of mannose residues is represented by letters instead of numbers. Thus, a branch containing one GlcNAc molecule would be represented by ‘1a’, while a branch containing one mannose residue would be represented by ‘Aa’. Higher letters correspond to higher numbers of mannose in the branch, i.e., B=2, C=3, D=4, etc. If no glycan is attached at a particular branch linkage it is represented as ‘0x’. The letter ‘u’ is reserved to depict monosaccharides that are attached in an unknown linkage. For the sixth digit-letter pair representing the bisecting GlcNAc there are only two possible values: ‘0x’ or ‘1a’ depending on whether there is a molecule attached or not. The final digit-letter pair is used to count the number of fucose residues attached to the core structure or any peripheral fucose attached to branch GlcNAc molecules. More details about the types of glycans that can be added to the structure are described hereinafter.

TABLE 1
Corresponding linkages and target position for each
of the seven digit-letter pairs of GlycoDigit

Linkage^{a}

| | Complex | High-mannose | |
|---|---|---|---|
| Position | branch | branch | Attached to |
| 1 | β1-2 | α1-2 | α1-3 linked mannose |
| 2 | β1-4 | α1-6 | α1-3 linked mannose |
| 3 | β1-2 | α1-3 | α1-6 linked mannose |
| 4 | β1-6 | α1-6 | α1-6 linked mannose |
| 5 | β1-4 | Not Available | α1-6 linked mannose |
| 6 | β1-4 | β1-4 | b1-4 linked mannose |
| 7 | α1-3/4/6 | α1-3/4/6 | core and peripheral |
| | | | GlcNAc |

^{a}GlcNAc, mannose or fucose residues can attach to the core structure through these linkages

GlcNAc, Galactose and Polylactosamine Chains

After a GlcNAc residue is added to the core structure, several other monosaccharides can sequentially be attached to it. Galactose (Gal) residues are attached to GlcNAc through a β1-4 link and the branch is then represented as ‘2a’ as listed in Table 2. This Galβ1-4GlcNAc structure is called a lactosamine unit and additional lactosamine units can attach to the first structure through a β1-3 link to form poly-lactosamine chains. The second embodiment of the GlycoDigit code allows up to four lactosamine units to be present in a single branch. Although the first GlcNAc and galactose moieties can be added individually, further additions are restricted in that they must be added together as a single lactosamine unit. This fact is reflected in Table 2 where digit values for branches with only lactosamine units are assigned to even numbers. Thus, a branch with two lactosamine units is depicted by ‘4a’; three units by ‘6a’, etc. Galactose can also attach to GlcNAc through a β1-3 link to form a neo-lactosamine unit (Varki et al). The GlycoDigit code does not allow repeating neo-lactosamine units and the first unit would be represented by ‘2b’ as listed in Table 2. The outermost galactose can have a final monosaccharide such as fucose or a sialic acid attached to it.

TABLE 2
Digit-letter values for different combinations
of GlcNAc and galactose chains

| Digit | Letter | Residue attached | Linkage |
|---|---|---|---|
| 1 | a | GlcNAc | β1-4 |
| 2 | a | Galactose | β1-4 |
| 2 | b | Galactose | β1-3 |
| 4 | a | Galβ1-4GlcNAc | β1-3 |
| 6 | a | Galβ1-4GlcNAc | β1-3 |
| 8 | a | Galβ1-4GlcNAc | β1-3 |

Terminal Residues

The outermost galactose residue in a branch can be capped by several terminal monosaccharides. Since even numbers are used to imply the presence of a galactose unit, odd numbers (3, 5, 7 and 9) are used to represent a different terminal sugar in the second embodiment of the GlycoDigit code. Table 3 lists the monosaccharides that can be added to the outermost galactose in several different linkage positions.

TABLE 3
Letter values for different combinations of
terminal sialic acid, fucose and galactose

| Letter | Residue Attached | Linkage |
|---|---|---|
| a | NeuNAc | α2-3 |
| b | NeuNAc | α2-6 |
| c | NeuNAc | Unknown |
| d | NeuGc | α2-3 |
| e | NeuGc | α2-6 |
| f | NeuGc | Unknown |
| g | Fucose | α1-2 |
| h | Galactose | α1-3 |

The digit value for these cases can be 3, 5, 7 or 9 depending on how many GlcNAc and galactose residues have been added in a branch

Sialic acids are the most common type of glycans added to the outermost galactose and are often attached either in α2-3 or α2-6 linkage. Though the sialic acid family is very diverse, N-acetyl-neuraminic acid (NeuNAc) and N-glycolyl-neuraminic acid (NeuGc) are the most common sialic acids observed. Mice produce glycoproteins almost exclusively with NeuGc, while CHO cells are a mix of mostly NeuNAc and a small amount of NeuGc (Baker K N, Rendall M H, Hills A E, Hoare M, Freedman R B, James D C (2001) “Metabolic control of recombinant protein N-glycan processing in NS0 and CHO cells.” Biotechnol Bioeng 73:188-202). NeuGc is absent in humans and glycoproteins containing it are actually immunogenic to humans (Irie A, Koyama S, Kozutsumi Y, Kawasaki T, Suzuki A (1998) “The molecular basis for the absence of N-glycolylneuraminic acid in humans.” J Biol Chem 273:15866-15871). In Table 3 the letters ‘a’ to ‘f’ are assigned to represent NeuNAc and NeuGc in various linkages. α2-8 linked sialic acids, which attach to α2-3 sialic acids, are currently not represented in the second embodiment of the GlycoDigit code.

Other terminal residues that can attach to the outermost galactose are fucose (represented by the letter ‘g’) and an additional α1-3 linked galactose (represented by the letter ‘h’). Fucose units attached to terminal galactose in the α1-2 linkage are found in some blood group antigens such as the Lewis Y and Lewis B antigens (Varki et al). The α1-3 galactosyl-transferase enzyme in mouse cells attaches an additional terminal galactose residue to the β1-4 linked galactose (Butler M (2006) “Optimisation of the cellular metabolism of glycosylation for recombinant proteins produced by mammalian cell systems.” Cytotechnology 50:57-76). This Galα1-3Galβ1-4GlcNAc structure is highly immunogenic in humans (Jenkins N, Parekh R B, James D C (1996) “Getting the glycosylation right: implications for the biotechnology industry.” Nat Biotechnol 14:975-981).

Fucosylation

The final digit-letter pair in the second embodiment of the GlycoDigit code is used to represent fucosylation on the core GlcNAc and on the outermost GlcNAc residues in branches attached to the core structure. Fucose is attached to the core GlcNAc residue through an α1-6 link while the peripheral fucosylation can occur through the α1-3 or α1-4 linkage (Ma B, Simala-Grant J L, Taylor D E (2006) “Fucosylation in prokaryotes and eukaryotes.” Glycobiology 16:158R-184R). It is important to note that this digit-letter pair only counts fucose molecules attached to GlcNAc and does not include fucose attached to the outermost galactose which is covered in the cases for representing terminal residues. The digit portion of the last digit-letter pair counts the number of fucose molecules attached to GlcNAc in the structure, while the letter is used to represent which branches are fucosylated and through which linkage. In order to keep the code as concise as possible, not all combinations of possible fucosylation sites are represented in the second embodiment of the GlycoDigit code. Only the outermost GlcNAc residue in a branch is allowed to be fucosylated. Additionally, if more than one branch is fucosylated then all fucose residues must be attached through the same type of linkage. Thus it is possible to have a structure with two fucose residues attached on the outer branches through α1-3 linkages, but not possible to have one fucose attached through an α1-3 link and the other through an α1-4 link. Table 4 lists all the combinations of fucosylation that can be represented by the second embodiment of the GlycoDigit code.

TABLE 4
Digit and letter values for the last digit-letter
pair in a GlycoDigit code, representing different
combinations of core and peripheral fucosylation

| Digit | Letter | Structure attached | Linkage |
|---|---|---|---|
| 1 | a | C^{a} | α1-6 |
| 1 | b | B1^{b} | α1-3 |
| 1 | c | B1 | α1-4 |
| 1 | d | B2 | α1-3 |
| 1 | e | B2 | α1-4 |
| 1 | f | B3 | α1-3 |
| 1 | g | B3 | α1-4 |
| 1 | h | B4 | α1-3 |
| 1 | i | B4 | α1-4 |
| 2 | a | C + B1 | α1-6 + α1-3 |
| 2 | b | C + B1 | α1-6 + α1-4 |
| 2 | c | C + B2 | α1-6 + α1-3 |
| 2 | d | C + B2 | α1-6 + α1-4 |
| 2 | e | C + B3 | α1-6 + α1-3 |
| 2 | f | C + B3 | α1-6 + α1-4 |
| 2 | g | C + B4 | α1-6 + α1-3 |
| 2 | h | C + B4 | α1-6 + α1-4 |
| 2 | i | B1 + B2 | α1-3 + α1-3 |
| 2 | j | B1 + B3 | α1-3 + α1-3 |
| 2 | k | B1 + B4 | α1-3 + α1-3 |
| 2 | l | B2 + B3 | α1-3 + α1-3 |
| 2 | m | B2 + B4 | α1-3 + α1-3 |
| 2 | n | B3 + B4 | α1-3 + α1-3 |
| 2 | o | B1 + B2 | α1-4 + α1-4 |
| 2 | p | B1 + B3 | α1-4 + α1-4 |
| 2 | q | B1 + B4 | α1-4 + α1-4 |
| 2 | r | B2 + B3 | α1-4 + α1-4 |
| 2 | s | B2 + B4 | α1-4 + α1-4 |
| 2 | t | B3 + B4 | α1-4 + α1-4 |
| 3 | a | C + B1 + B2 | α1-6 + α1-3 + α1-3 |
| 3 | b | C + B1 + B3 | α1-6 + α1-3 + α1-3 |
| 3 | c | C + B1 + B4 | α1-6 + α1-3 + α1-3 |
| 3 | d | C + B2 + B3 | α1-6 + α1-3 + α1-3 |
| 3 | e | C + B2 + B4 | α1-6 + α1-3 + α1-3 |
| 3 | f | C + B3 + B4 | α1-6 + α1-3 + α1-3 |
| 3 | g | C + B1 + B2 | α1-6 + α1-4 + α1-4 |
| 3 | h | C + B1 + B3 | α1-6 + α1-4 + α1-4 |
| 3 | i | C + B1 + B4 | α1-6 + α1-4 + α1-4 |
| 3 | j | C + B2 + B3 | α1-6 + α1-4 + α1-4 |
| 3 | k | C + B2 + B4 | α1-6 + α1-4 + α1-4 |
| 3 | l | C + B3 + B4 | α1-6 + α1-4 + α1-4 |
| 3 | m | B1 + B2 + B3 | α1-3 + α1-3 + α1-3 |
| 3 | n | B1 + B2 + B4 | α1-3 + α1-3 + α1-3 |
| 3 | o | B1 + B3 + B4 | α1-3 + α1-3 + α1-3 |
| 3 | p | B2 + B3 + B4 | α1-3 + α1-3 + α1-3 |
| 3 | q | B1 + B2 + B3 | α1-4 + α1-4 + α1-4 |
| 3 | r | B1 + B2 + B4 | α1-4 + α1-4 + α1-4 |
| 3 | s | B1 + B3 + B4 | α1-4 + α1-4 + α1-4 |
| 3 | t | B2 + B3 + B4 | α1-4 + α1-4 + α1-4 |
| 4 | a | C + B1 + B2 + B3 | α1-6 + α1-3 + α1-3 + α1-3 |
| 4 | b | C + B1 + B2 + B4 | α1-6 + α1-3 + α1-3 + α1-3 |
| 4 | c | C + B1 + B3 + B4 | α1-6 + α1-3 + α1-3 + α1-3 |
| 4 | d | C + B2 + B3 + B4 | α1-6 + α1-3 + α1-3 + α1-3 |
| 4 | e | C + B1 + B2 + B3 | α1-6 + α1-4 + α1-4 + α1-4 |
| 4 | f | C + B1 + B2 + B4 | α1-6 + α1-4 + α1-4 + α1-4 |
| 4 | g | C + B1 + B3 + B4 | α1-6 + α1-4 + α1-4 + α1-4 |
| 4 | h | C + B2 + B3 + B4 | α1-6 + α1-4 + α1-4 + α1-4 |
| 4 | i | B1 + B2 + B3 + B4 | α1-3 + α1-3 + α1-3 + α1-3 |
| 4 | j | B1 + B2 + B3 + B4 | α1-4 + α1-4 + α1-4 + α1-4 |
| 5 | a | C + B1 + B2 + B3 + B4 | α1-6 + α1-3 + α1-3 + α1-3 + |
| | | | α1-3 |
| 5 | b | C + B1 + B2 + B3 + B4 | α1-6 + α1-4 + α1-4 + α1-4 + |
| | | | α1-4 |

^{a}C implies that the fucose is attached to the core GlcNAc
^{b}B indicates which branch's outermost GlcNAc is fucosylated

Results

Representing N-Linked Glycans with the GlycoDigit Code

The GlycoDigit code can be used to represent complex, high-mannose and hybrid type N-linked glycans. FIGS. 4a-4c depict three different N-linked glycan structures of different sub-types and their corresponding representation using the first embodiment of the GlycoDigit code, and FIGS. 5a-5c depict three different glycan structures and their corresponding representation in the second embodiment of the GlycoDigit code. In all of FIGS. 4a-4c and 5a-5c; circled numbers depict the branch position; un-circled numbers define the terminal monosaccharide of each branch; and the underlined alpha-numeric code is the GlycoDigit code representation for each structure. The shaded portion in FIGS. 4a-4c is the core structure common to all N-linked glycans.

FIG. 4a is a complex type N-linked glycan with the following digits for the code:

1st digit=7: The branch terminates in NeuNAc (N-acetylneuraminic acid)

2nd digit=3: The branch terminates in GlcNAc (N-acetylglucosamine)

3rd digit=5: The branch terminates in Galactose

4th digit=1: There is a non-existent branch

5th digit=1: No bisecting GlcNAc is attached in this branch

6th digit=3: Fucose is attached in this structure

Thus the final code for the structure in FIG. 4a is (7 3 5 1 1 3). The detailed linkage information of the monosaccharides attached in each branch can be deduced by looking up the digit value in Table I. The code for a high-mannose type glycan structure is shown in FIG. 4b. The value for each digit is based on the number of mannose residues attached at each branch. It is important to note that this format allows a maximum of nine mannose residues to be attached in a structure, as is the case for secreted mammalian glycoproteins, as described hereinafter. The structure in FIG. 4b contains this maximum permissible amount of mannose. A hybrid glycan structure and its corresponding code are shown in FIG. 4c. As described in Methods, branches 1 and 2, and branches 3 and 4 in a tetra-antennary N-linked glycan must be of the same type respectively, i.e. either both mannose, or both complex type. For example, it is not possible to have branch 1 with a mannose residue and branch 2 with a GlcNAc residue.

The rules described herein are not intended to cover the N-linked glycan structures for all species. Some vertebrate structures have been observed to have five branches, a third branch attached to the upper core mannose (Varki et al.). In CHO cells, a similar branch has been observed to be present only as an intermediary step in the glycosylation pathway (Butler M. 2006. “Optimisation of the cellular metabolism of glycosylation for recombinant proteins produced by mammalian cell systems.” Cytotechnology, 50:57-76). In addition, several other variations on possible linkages have been observed in other species (Schachter H, Brockhausen I, Hull E. 1989. “High-performance liquid chromatography assays for N-acetylglucosaminyltransferases involved in N- and O-glycan synthesis.” Methods Enzymol., 179:351-397). Nevertheless, the GlycoDigit code is sufficiently applicable to most mammalian species that are commonly used in the production of recombinant proteins.

The first embodiment of the GlycoDigit code provides a simple means for generating all possible glycan structures. For branches 1 to 4 there are 10 possible alpha-numeric characters that can be used to describe the branch structure (1, 3, 5, 7, A, B, C, D, E and F), while there are two possible numbers for the 5th and 6th branch (1, 3). Thus, 10×10×10×10×2×2=40,000 different structures can be generated and represented in the six digit-letter pair embodiment of the GlycoDigit code. However, not all of these structures are valid. Invalid structures can be filtered out by the rules described hereinafter, thus resulting in 4860 N-linked glycan structures that can be considered as theoretically valid glycan structures in the six character alpha-numeric embodiment of the GlycoDigit code. Of course, it is possible to further refine the rules to give rise to the glycan population pertaining to the appropriate mammalian cell line.

Table 5 summarizes the definition for each digit in the first (six character alpha-numeric) embodiment of the GlycoDigit code, and also shows the full branch structure and the anomeric linkage information. Blank cells indicate that the value is not possible for that digit position.

TABLE 5
Definition of digit values for the corresponding
monosaccharide and linkage information.

| Digit | Terminal | | | |
|---|---|---|---|---|
| Value | Monosaccharide | 1^{st}-4^{th} digit | 5^{th} digit | 6^{th} digit |
| 1 | Non- | Non- | Non- | Non- |
| | existence | existence | existence | existence |
| 3 | GlcNAc | GlcNAc- | GlcNAc- | Fucose |
| 5 | Galactose | Galβ1-4GlcNAc | — | — |
| 7 | NeuNAc | NeuNAcα2-3Galβ1-4GlcNAc- | — | — |
| A | Mannose | Man- | — | — |
| B | Mannose | Manα1-2Man- | — | — |
| C | Mannose | Manα1-2Manα1-2-Man- | — | — |
| D | Mannose | Manα1-2Manα1-2Manα1-2- | — | — |
| | | Man- | | |
| E | Mannose | Manα1-2Manα1-2Manα1- | — | — |
| | | 2Manα1-2-Man- | | |
| F | Mannose | Manα1-2Manα1-2Manα1- | — | — |
| | | 2Manα1-2Manα1-2-Man- | | |

Summary of all possible values for designated digit positions with defined antennary by corresponding digit positions. Blank cells indicate that value is not possible for that digit position

Three additional rules are defined to describe the N-linked glycan structures of secreted proteins from CHO cells by the six character alpha-numeric embodiment of the GlycoDigit code.

Rule 1: For high-mannose and hybrid subtypes in secreted mammalian cells, the maximum possible number of mannose residues attached to the core structure is six, making the total number of mannose residues in a structure equal to nine (counting the three residues in the trimannosyl core) (Varki et al.).

Rule 2: The six character alpha-numeric embodiment of the GlycoDigit code only allows six mannose at most in a single branch.

Rule 3: For hybrid structures branches 1 and 2 and branches 3 and 4 must be of the same type respectively, i.e., either both mannose, or both complex type.

The complex type glycan structure in FIG. 5a is a tri-antennary structure with a Lewis Y type epitope attached on the branch connected to the α1-3 linked mannose. In the seven digit-letter pair embodiment, the GlycoDigit code for this structure is [0x 3g 1a 3a 0x 0x 2c]. The Man₉GlcNAc₂ structure in FIG. 5b is a high mannose structure that is the starting point for all further glycosylation reactions in the endoplasmic reticulum and Golgi apparatus. Since mannose residues are represented by letters instead of numbers the corresponding code for this structure is [Ba 0x Ba Ba 0x 0x 0x]. A hybrid structure is shown in FIG. 5c with two high-mannose branches and two complex branches. A sialyl Lewis X structure is present in the first complex branch with a fucose residue attached to the branch GlcNAc, while a di-lactosamine chain is shown in the second branch. As shown in the figure, this structure is represented by the GlycoDigit code as [3a 4a Aa Ba 0x 1a 2a].

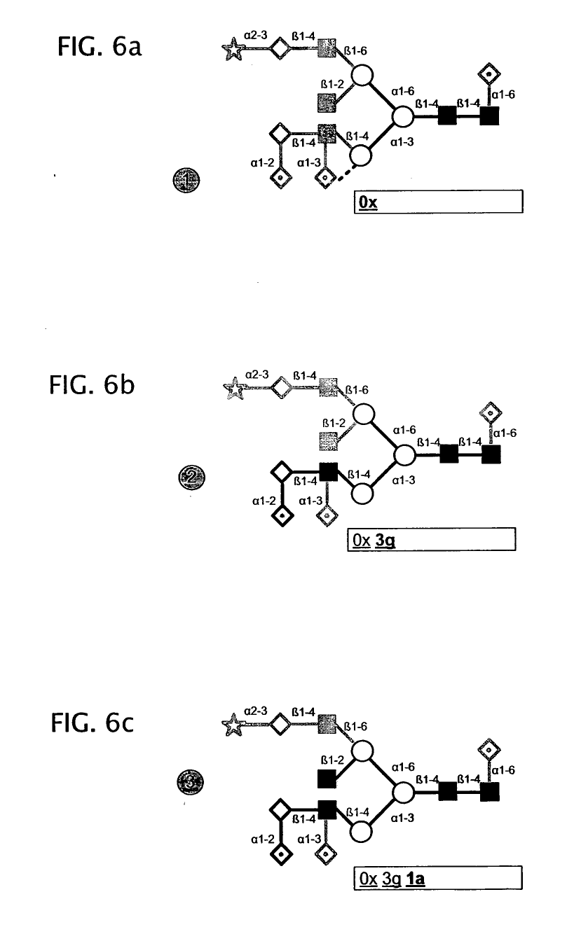

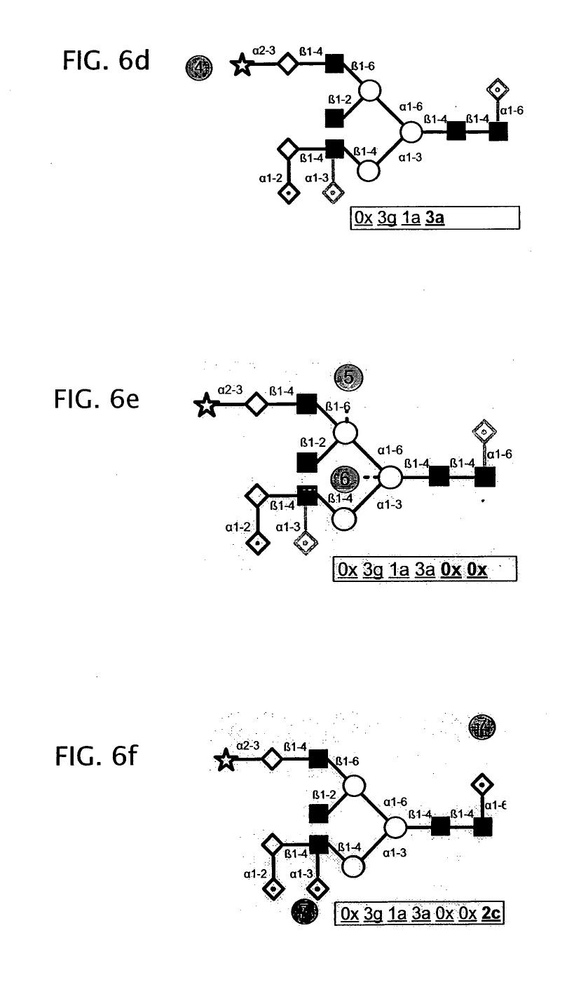

FIGS. 6a-6f illustrate a step-by-step representation of the corresponding GlycoDigit code (seven digit-letter embodiment) for the complex type structure presented in FIG. 5a. Each digit-letter pair can be coded as follows:

Starting from the first digit-letter pair, in this case the corresponding branch is empty and so the representation is ‘0x’.

Looking at the second branch attached to the α1-3 core mannose, it has three residues and ends in a terminal fucose; its representation is ‘3g’ as listed in Table 3.

The branch in the third digit-letter position has one GlcNAc residue and is represented as ‘1a’.

The fourth branch has three residues ending in an α2-3 linked sialic acid. The code for this branch is ‘3a’.

The fifth and sixth branches are empty and thus both are represented by ‘0x’.

The value for the last digit-letter position is ‘2c’ since in addition to the core fucose, there is also a fucose residue attached to the GlcNAc in the second branch in an α1-3 linkage (see Table 4). The fucose attached to the galactose in that branch is represented in the code for the second branch and is not counted here.

Thus the code for the entire structure results in [0x 3g 1a 3a 0x 0x 2c].

It should be noted that the GlycoDigit code does not aim to provide comprehensive coverage of all possible glycan structures found in all species. Instead it focuses primarily on structures found in secreted glycoproteins in mammalian cell lines such as CHO cells, while still remaining extensible. For this reason the seven digit-letter pairs are chosen to represent the six linkage sites on the core structure for GlcNAc residues along with the ability to describe attached fucose molecules. Currently the GlycoDigit code can represent structures with mannose, GlcNAc, galactose, fucose and sialic acid residues present in them. It can distinguish between NeuNAc and NeuGc; and is capable of representing terminal galactose and fucose. Several structures that are not naturally expressed in CHO cells have been produced in engineered CHO cell lines. These include bisecting GlcNAc (Sburlati et al; Umana et al] repeating lactosamine chains (Sasaki H, Bothner B, Dell A, Fukuda M (1987) “Carbohydrate structure of erythropoietin expressed in Chinese hamster ovary cells by a human erythropoietin cDNA.” J Biol Chem 262:12059-12076) and Lewis blood group structures (Thomas L J, Panneerselvam K, Beattie D T, Picard M D, Xu B, Rittershaus C W, Marsh Jr H C, Hammond R A, Qian J, Stevenson T, Zopf D, Bayer R J (2004) “Production of a complement inhibitor possessing sialyl Lewis X moieties by in vitro glycosylation technology.” Glycobiology 14:883-893; Barrabés S, Pagès-Pons L, Radcliffe C M, Tabarès G, Fort E, Royle L, Harvey D J, Moenner M, Dwek R A, Rudd P M, De Llorens R, Peracaula R (2007) “Glycosylation of serum ribonuclease 1 indicates a major endothelial origin and reveals an increase in core fucosylation in pancreatic cancer.” Glycobiology 17:388-400).

With respect to the second embodiment, if additional branches are required to cover other cases, more digit-letter pairs can be added to the code to represent them. Further, the index-based letters for representing additional linkage information allow the easy addition of further linkage and residue type options. Conversely, the code can be simplified in cases where there are fewer than seven branches or if linkage information is not needed. The main emphasis in the GlycoDigit code is on the fact that the code keeps a numeric component, which can serve as the basis for several computational applications.

Applications of the GlycoDigit Code

Comparing Glycan Structures

The development of BLAST (Altschul S F, Gish W, Miller W, Myers E W, Lipman D J (1990) “Basic local alignment search tool.” J Mol Biol 215:403-410) (“Altschul et al”) provided a solution to a fundamental question that biologists had been asking, i.e., how to measure similarity between different sequences of nucleotides and proteins. However, such algorithms are not directly applicable to the comparison of glycans due to their tree-like structure. Recently a few techniques have been developed for comparing glycans (Aoki K F, Yamaguchi A, Ueda N, Akutsu T, Mamitsuka H, Goto S, Kanehisa M (2004) “KCaM (KEGG Carbohydrate Matcher): a software tool for analyzing the structures of carbohydrate sugar chains.” Nucleic Acids Res 32:W267-272 (“Aoki et al”); Aoki K F, Mamitsuka H, Akutsu T, Kanehisa M (2005) “A score matrix to reveal the hidden links in glycans.” Bioinformatics 21:1457-1463) but this research area is still in its infancy. In both the six- and seven digit-letter pair embodiments of the GlycoDigit code, we define a difference operator, which allows for easy comparison of different glycan structures.

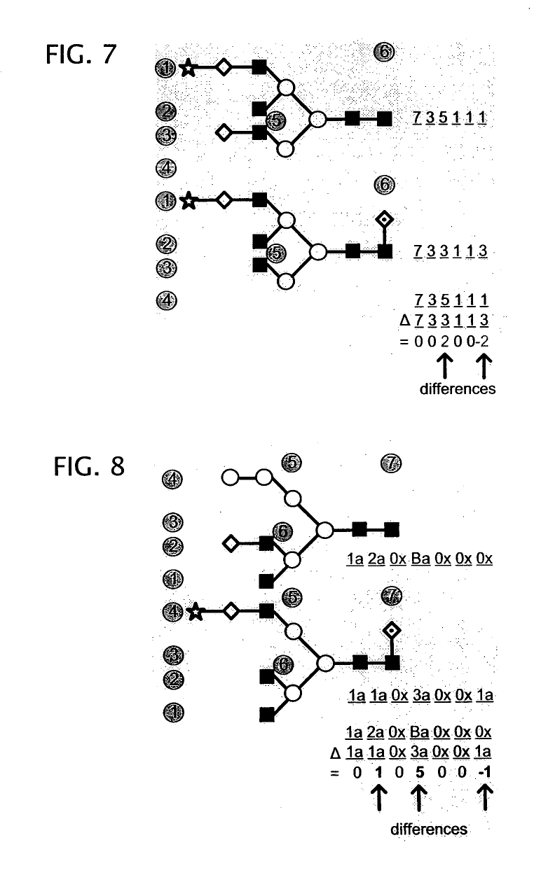

FIG. 7 depicts complex and hybrid N-linked glycan structures and their corresponding GlycoDigit codes for the six character alpha-numeric embodiment of the GlycoDigit code. There are two differences between the structures; the first one is missing a fucose residue attached to branch 6 while the second structure does not have the galactose residue attached to branch 3. The difference between the structures is obtained as (0 0 2 0 0 −2). The resulting code is not a valid glycan structure, but provides information about the difference between the two input structures. Zero values indicate that branches on both of the structures are exactly same, while non-zero values mean the branches are different. Even numbers imply that both branches being compared are of the same type, either both complex or both high-mannose. An odd number would imply that a complex branch is being compared to a high-mannose branch. The result from the above example verifies that there are differences between the two structures in the 3rd and 6th branch.

A lookup table (Table 6) is defined to use the results from the difference operator to find the specific residue and linkage differences between structures. For each branch being compared, the larger digit from the two input structure is indexed against all possible resulting differences. Considering only complex type structures for example, a branch with the value 7 (NeuNAc) can only be compared against the values 7 (NeuNAc), 5 (Gal), 3 (GlcNAc), and 1, meaning that the resulting differences can only be 0, ±2, ±4, and ±6 (see Difference column in Table 6). The zero value indicates no change, and is not recorded in the lookup table. For each of these possible differences, the table lists the linkages that must be changed in order to get from the first to the second structure. For positive differences, linkages must be removed, while for negative values linkages are added. Table 6 is the lookup table for complex N-linked glycan comparisons between single branches. Using the result code obtained in FIG. 7, the exact differences between the two structures can be found. Considering the digits in each structure for the 3rd branch we can see that the larger of the two digits is 5, and the difference value is 2. The corresponding highlighted cell in the lookup table shows that GlcNAc residue attached via the β1→4 linkage is removed in the second structure. Similarly for the 6th branch, it can be shown that a fucose residue has been added via the α1→6 linkage.

TABLE 6
A condensed version of the lookup table for the comparison of branches in
complex N-linked glycan structures

| | | Num | | | | |
|---|---|---|---|---|---|---|
| Larger | | Reaction | Linkage Changes | | | |
| Digit | Difference | Steps | 1^{st} & 3^{rd} digit | 2^{nd} & 4^{th} digit | 5^{th} digit | 6^{th} digit |
| 7 | 6 | 3 | α2→3(−) | α2→3(−) | N/A | N/A |
| | | | β1→4(−) | β1→4(−) | | |
| | | | β1→4(−) | β1→2(−) | | |
| | 4 | 2 | α2→3(−) | α2→3(−) | N/A | N/A |
| | | | β1→4(−) | β1→4(−) | | |
| | 2 | 1 | α2→3(−) | α2→3(−) | N/A | N/A |
| | −2 | 1 | α2→3(+) | α2→3(+) | N/A | N/A |
| | −4 | 2 | β1→4(+) | β1→4(+) | N/A | N/A |
| | | | α2→3(+) | α2→3(+) | | |
| | −6 | 3 | β1→4(+) | β1→2(+) | N/A | N/A |
| | | | β1→4(+) | β1→4(+) | | |
| | | | α2→3(+) | α2→3(+) | | |
| 5 | 4 | 2 | β1→4(−) | β1→4(−) | N/A | N/A |
| | | | β1→4(−) | β1→2(−) | | |
| | 2 | 1 | 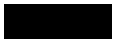 | β1→4(−) | N/A | N/A |
| | −2 | 1 | β1→4(+) | β1→4(+) | N/A | N/A |
| | −4 | 2 | β1→4(+) | β1→4(+) | N/A | N/A |
| | | | β1→4(+) | β1→2(+) | | |
| 3 | 2 | 1 | β1→4(−) | β1→2(−) | β1→2(−) | α1→6(−) |
| | −2 | 1 | β1→4(+) | B1→2(+) | β1→2(+) | 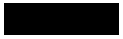 |

Lookup Table 6 also contains information on the number of reaction steps necessary for the difference between individual branches between the structures. The number of required reaction steps for each branch can be obtained by dividing the absolute value of the difference between two branches by 2. For the above example two reactions steps must take place to convert the first structure into the second one, i.e. the removal of the GlcNAc residue and the addition of fucose.

The full lookup table also contains information on the changes that occur when comparing branches where both inputs are of the high-mannose type. For example, in comparing the two branches of a high-mannose structure with digits B (value of 4) and D (value of 8) the difference would be 4 and can be described as adding two mannose residues to the first structure. The comparison between complex and high-mannose branches in hybrid glycan structures is more complicated. In order to convert a high-mannose structure to a complex one, all of the mannose residues must be removed before any other monosaccharides can be attached. Comparing branches represented by the digits C and 7 would imply that the three mannose residues have to be removed and that a GlcNAc, galactose and NeuNAc had to be added in a total of six reaction steps.

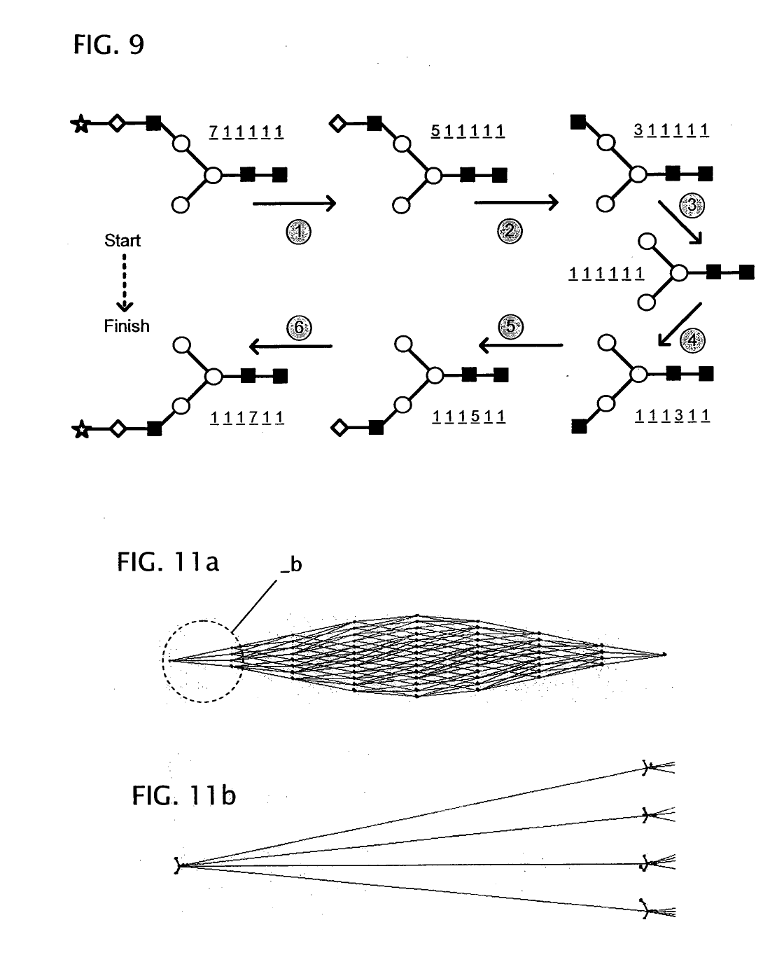

FIG. 8 depicts complex and hybrid N-linked glycan structures and their corresponding GlycoDigit codes for the seven letter-digit pair embodiment. There are three differences between the structures: the first one is the missing fucose residue attached to the core GlcNAc; the second is the missing galactose residue in lower branch; and finally the fourth branch is of different types in the two structures. As shown in FIG. 8, the difference between the structures is obtained as [0 1 0 5 0 0 −1]. The difference operator only compares the digit values in the code and ignores the letter values. As such, the resulting code provides information about the difference between the two structures. Zero values indicate that branches on both of the structures are exactly same, while non-zero values mean the branches are different. A special case arises when high-mannose branches are compared against complex branches. In this situation, the difference between branches is defined as the sum of the two digit values for that branch. The result from the above example verifies that there are differences between the two structures in the second, fourth and seventh branch positions.

The result code from the difference operator can be used to calculate the number of reaction steps necessary to convert one structure to another for the seven digit-letter pair embodiment. Adding the absolute values of the digits in the difference code reveals the number of reactions needed to convert the first structure into the second. From the difference code, we can calculate the number of steps to be 7 (0+1+0+5+0+0+1). In the case of two complex branches being compared if the difference digit for that branch is positive then it implies that glycans must be added as part of the conversion, while a negative difference means glycans must be removed. The comparison between complex and high-mannose branches in hybrid glycan structures is more complicated. In order to convert a high-mannose branch to a complex one, all of the mannose residues must first be removed before any other monosaccharides can be attached. Comparing the fourth branch represented by the digits B and 3 in the two structures respectively would imply that the two mannose residues have to be removed and that a GlcNAc, galactose and NeuNAc have to be added for a total of five reaction steps. Tables 1 through 3 can be used to find out which monosaccharide is added for each digit and in which linkage. This information can be used in reverse to find out which linkages are removed when converting one structure to another.

A Distance Measurement Between Two N-Linked Glycan Structures

Equation (1) represents an algorithm for comparing two valid glycan structures in terms of reaction distance, for the six character alpha-numeric embodiment of the GlycoDigit code:

$$\% Nearness = \frac{( max\_possible \_reactions - total\_reactions )}{max\_possible \_reactions} \times 100 \qquad Eq . ( 1 )$$

Using this algorithm, the nearness score between two structures can be simply calculated, allowing the determination of the number of reaction steps needed to convert one structure to another, as described hereinafter. It should be noted that the score is just a naïve approximation, and does not have any clear biological significance.

FIG. 9 shows two glycans and the reaction steps needed to convert one structure to another. The structures are represented by the codes (7 1 1 1 1 1) and (1 1 1 7 1 1), with a similarity score of 84.2%.

For the first four branches, the maximum number of reactions needed to convert a branch with six mannose residues into a branch with a terminal NeuNAc residue is nine reactions. Therefore, the maximum number of possible reactions would be (9×4) plus one reaction each for the bisecting GlcNAc at branch 5 and the fucose at branch 6 i.e: 38 possible reactions. The score can then be defined as

$$\% Nearness = \frac{( 38 - total\_reactions )}{38} \times 100 \qquad Eq . ( 2 )$$

Using the first and last two structures in FIG. 7 as an example, the difference in terms of reaction steps between the two structures is 2. Therefore the nearness between the two structures can be calculated to be

$$\% Nearness = \frac{( 38 - 2 )}{38} \times 100 = \frac{36}{38} \times 100 = 94.7 \% \qquad Eq . ( 3 )$$

Six reaction steps are needed to convert the first structure of FIG. 9 to the last one. Therefore, the nearness between the first and last structures of FIG. 9 can be calculated using Equation (1) to be 84.2%. However, these structures are only intermediate and the final structure is always valid. Note that the first structure and the final converted structure in FIG. 9 are isomers of each other and may be biologically indistinguishable, a fact not represented by the 84.2% similarity score. Further work is needed to establish a more biologically relevant scoring system. A web based graphical interface has been developed to implement the current algorithm and provide intuitive results, as described hereinafter.

Constructing Glycosylation Networks

The glycosylation reaction network can be thought of as a graph with the nodes representing glycan structures and edges showing possible enzymatic reactions. A single glycan structure can act as a substrate to multiple reactions and also be the end product of several reactions, thus creating a highly branched network. Another characteristic feature of the glycan network is how any intermediary structure can be considered an end product and lead to the large variety of structures seen in natural systems. Visualizing such a network can improve our understanding of the glycosylation pathway and serve as a basis for in silico experiments.

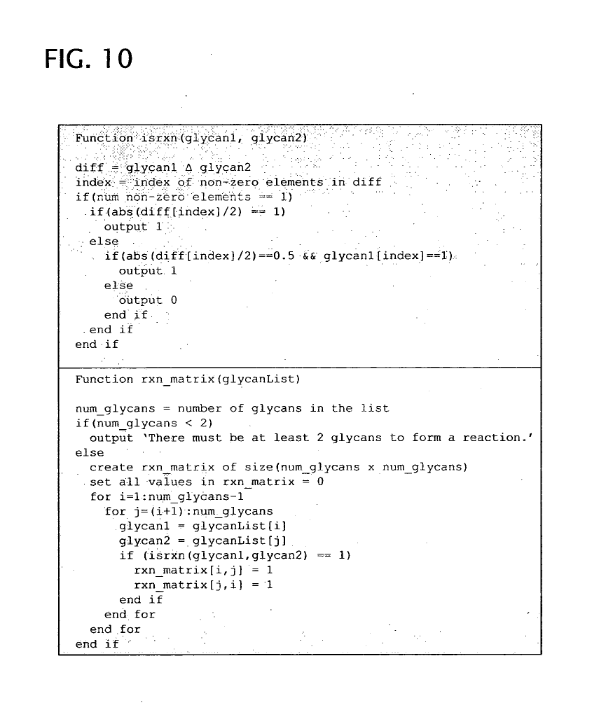

To ease storage and processing, a symmetric adjacency matrix was created to store the reaction pairs. A 5100×5100 matrix was created with each (i, j) value recording whether glycan i reacts with glycan j. A zero value implies there is no reaction between these two glycans, while a value of 1 means that there is a reaction link. The difference operator as described above in connection with the first embodiment was used in creating a pair of functions which populate the adjacency matrix; these functions were implemented in MATLAB and their corresponding pseudocode versions are shown in FIG. 10. The function isrxn takes two glycan structures as input and returns 1 if there is one and only one reaction needed to convert one structure to the other. The full list of glycan structures is passed to the rxn_matrix function, which creates the adjacency matrix and populates it with 1's each time there is a reaction between two glycans.

In order to visualize the glycosylation network, glycans were arranged from the basic core structure and sugar residues were added until the structure was fully sialylated. Glycans were classified into groups based on the number of reaction steps that separated each glycan from the core structure. For the case of complex type glycans, the core structure would be represented as 111111 in the first embodiment of the GlycoDigit code, while the end point would be a fully sialylated structure represented by the code 777733. The visualization algorithm draws the individual glycan structures in each group and then draws lines between those structures that have a reaction link.

Two data sets of glycan structures were created to test the visualization algorithm. The first set was the full 5100 theoretical glycans generated by GlycoDigit with 19372 reaction pairs. A much smaller data set comprising only 64 structures and 160 reactions was also created that only contained those complex type glycans with only two of the first four branches present. In both cases the resulting network showed a highly branched tree structure that diverged at first and then converged. At the start of the network there are many possible sites to attach sugars which leads to the divergent nature, but as these fill up the number of possible choices decreases and the network converges to the final few structures. The first network showed a tree structure with a depth of 15 levels, while the smaller set had a depth of 9. The number of glycans and reactions in each level for both cases are summarized in Table 7. FIGS. 11a and 11b show the network distribution for the second data set.

TABLE 7
Number of glycan structures and reactions in
each level of the network for both data sets.

| | Number of | | Number of | Number of |
|---|---|---|---|---|
| | glycan | Number of | glycans in | reactions in |
| | structures in | reactions in | reduced | reduced |
| Level | full set | full set | data set | data set |
| 1 | 1 | 10 | 1 | 4 |
| 2 | 10 | 74 | 4 | 14 |
| 3 | 41 | 264 | 8 | 26 |
| 4 | 112 | 668 | 12 | 36 |
| 5 | 240 | 1340 | 14 | 36 |
| 6 | 424 | 2232 | 12 | 26 |
| 7 | 644 | 3000 | 8 | 14 |
| 8 | 784 | 3164 | 4 | 4 |
| 9 | 761 | 2934 | 1 | 0 |
| 10 | 670 | 2498 | 0 | 0 |
| 11 | 539 | 1744 | 0 | 0 |
| 12 | 356 | 964 | 0 | 0 |
| 13 | 189 | 394 | 0 | 0 |
| 14 | 74 | 86 | 0 | 0 |
| 15 | 15 | 0 | 0 | 0 |

A list of enzymes involved in the addition and removal of monosaccharide units to the glycan structure were obtained from KEGG (Kanehisa M., Goto S., Hattori M., Aoki-Kinoshita K. F., Itoh M., Kawashima S., Katayama T., Araki M., and Hirakawa M. “From genomics to chemical genomics: new developments in KEGG.” Nucleic Acids Res., 34:D354-357, 2006). 5100 theoretical glycans of all three subtypes were obtained from the first embodiment of the GlycoDigit code, and 19372 reaction pairs were created for pairs of glycan structures that were linked together through an enzymatic reaction.

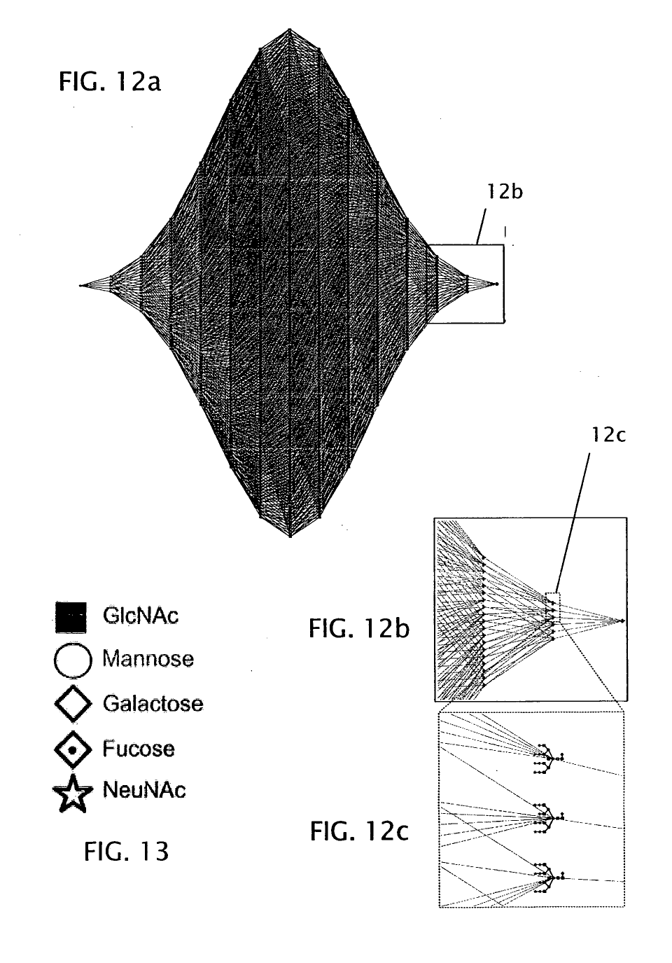

Using the numeric index of the second embodiment of the GlycoDigit code, an N-linked glycosylation network was constructed that can be represented as a graph with the nodes and edges corresponding to glycan structures and reaction steps, respectively, as shown in FIGS. 12a-12c.

Using the second embodiment of the GlycoDigit code, we enumerated all possible complex type glycan structures commonly secreted in CHO cells, starting from the core structure, which is represented as [0x 0x 0x 0x 0x 0x 0x]. This enumeration was simply carried out by incrementing each digit in the GlycoDigit code by 1, indicating that sugar residues such as GlcNAc, galactose, fucose and sialic acid are sequentially attached to the core structure through enzyme processing by relevant glycosyltransferases. This process continued until the glycan became a tetra-antennary fully sialylated structure with core fucosylation, represented by the code [3a 3a 3a 3a 0x 1a 1a], thus generating 1024 complex type glycans and 4096 reaction steps each linking two subsequent glycans.

In order to visualize the constructed network, the resulting graph was arranged in a hierarchical manner. First, all glycans were classified into different hierarchical layers based on the number of sugars attached. The core structure [0x 0x 0x 0x 0x 0x 0x] was initiated as the first layer, followed by the second layer composed of glycans that had added one sugar each to the core structure and so on until last layer containing a fully sialylated glycan structure [3a 3a 3a 3a 0x 1a 1a]. Once all glycans are placed in their corresponding layers, associated reaction edges linking glycan pairs are visualized within the network graph. FIGS. 12a-12c illustrate the resulting network, which is a highly branched structure in which individual glycan structures are represented as nodes in the network while edges represent enzymatic reaction steps between two glycans. It should be noted that the current network is an approximation of the glycosylation pathway in CHO cells since the enzymatic requirements and restrictions (Hossler P, Goh L T, Lee M M, Hu W S (2006) “GlycoVis: visualizing glycan distribution in the protein N-glycosylation pathway in mammalian cells.” Biotechnol Bioeng 95:946-960 (Hossler et al I″) were not fully considered during the network construction.

Most biological pathways are often complex and visualizing their structure is one of the most useful steps in studying them. The networks described herein can be used to identify possible pathways to link glycan structures, or find shorter paths than were previously known. In the current model there are often several possible pathways to get from one structure to another, but these paths might not always be biologically plausible. Depending on which species is being modeled, additional rules of which glycans can actually react to form others can be incorporated to make the network more realistic. The modular nature of the algorithms allows users to define their own model of reaction pairs and visualize them.

Metabolic flux analysis is one application that greatly benefits from the presence of a visual interface. Additional information can be added to the data model to allow in silico re-engineering of the pathway. The visualization system provides a good basis for building models for this kind of analysis. It can be implemented with an interactive user interface to incorporate experimental data and provide a web browser based service.

Discussion

Research in glycome informatics is slowly catching up with the progress that has been made in other ‘omics’ areas. As described herein, the GlycoDigit code in accordance with the present invention is based on a pre-defined branching structure of N-linked glycans that are commonly found in most mammalian cells. Compared to other standard text representations for glycans, the GlycoDigit code is much shorter and more intuitive as it focuses on branches instead of previous methods describing individual monosaccharide units. For example, the glycan structure illustrated in various formats in FIG. 2 is simply coded as [0x 2a 1a 3a 0x 0x 1a] by the seven-digit embodiment of the GlycoDigit code to represent its structure. A shorter representation is easier to enter manually and is not as susceptible to typographical or formatting errors unlike other longer and text-based standards.

Although the GlycoDigit code may be unable to provide comprehensive coverage of all possible glycan structures, it is adaptable and can be customized according to the user's requirements. For example, the number of branches allowed in a structure can be increased or decreased by adjusting the number of digit-letter pairs, while more choices can be added to the letter index to represent different linkage information. The GlycoDigit code is also interoperable, which allows it to be incorporated into a laboratory glyco-information management system in a retrievable format, thereby providing useful resources for biomedical and biotechnological applications (Hashimoto K, Goto S, Kawano S, Aoki-Kinoshita K F, Ueda N, Hamajima M, Kawasaki T, Kanehisa M (2006) “KEGG as a glycome informatics resource.” Glycobiology 16:63R-70R; Lutteke T, Bohne-Lang A, Loss A, Goetz T, Frank M, von der Lieth C W (2006) “GLYCOSCIENCES.de: an Internet portal to support glycomics and glycobiology research.” Glycobiology 16:71R-81R; Raman R, Venkataraman M, Ramakrishnan S, Lang W, Raguram S, Sasisekharan R (2006) “Advancing glycomics: implementation strategies at the consortium for functional glycomics.” Glycobiology 16:82R-90R). As such, relevant glycan structures can be easily stored, accessed, retrieved and rapidly converted into their pictorial formats.

Research on the glycosylation pathway to control the diversity of glycosylation is another area that can benefit from the GlycoDigit code. A simplified numeric representation instead of a text-based representation of glycan structures can further advance the development of computer aided analysis tools to study such a complex network (Hossler et al I). The format of the GlycoDigit code as described herein can be easily applied to constructing and visualizing networks of glycan interactions. This applicability may not be provided as easily by text-based representations. Moreover, describing differences between glycans in terms of reaction steps and having an exhaustive list of possible glycan structures as illustrated in FIGS. 8a-8c will provide the basis for developing mathematical models of the glycosylation pathway (Hossler P, Mulukutla B C, Hu W S (2007) “Systems analysis of N-glycan processing in mammalian cells.” PLoS ONE 2(8):e713; Krambeck F J, Betenbaugh M J (2005) “A mathematical model of N-linked glycosylation.” Biotechnol Bioeng 92:711-728; Umana P, Bailey J E (1997) “A mathematical model of N-linked glycoform biosynthesis.” Biotechnol Bioeng 55:890-908).

Further work is needed to define a biologically meaningful measure of similarity among glycan structures in the context of the GlycoDigit code. As was the case with protein structures, it is expected that a similarity of glycan structures will imply a similarity of function as well (Altschul et al; Aoki et al; Bertozzi C R, Kiessling L L (2001) “Carbohydrates and glycobiology review: chemical glycobiology.” Science 291:2357-2364). The GlycoDigit code in accordance with the present invention is also extendable to allow the representation of a more varied range of N-linked glycan structures.

Modifications and variations of the above-described embodiments of the present invention are possible, as appreciated by those skilled in the art in light of the above teachings. It is therefore to be understood that, within the scope of the appended claims and their equivalents, the invention may be practiced otherwise than as specifically described.

1. A system for representing at least a portion of an oligosaccharide, the system comprising a fixed-length alpha-numeric code, wherein the code represents the number and position residues attached to the oligosaccharide, wherein the numeric portion of each alphanumeric pair corresponds to the number of residues attached to the oligosaccharide represented by the alpha-numeric pair; and the alpha portion of each alpha-numeric pair serves as an index to a table containing additional information about the type of linkage and the specific residue added.

2. The system according to claim 1, further comprising an information management system incorporating the code in a retrievable format.

3. The system according to claim 1, wherein the oligosaccharide is an N-linked glycan structure.

4. The system according to claim 3, wherein the N-linked glycan structure is one of a complex, high-mannose and hybrid type.

5. The system according to claim 1, wherein the residues are selected from the group consisting of mannose, N-acetylglucosamine, galactose, fucose and sialic acid residues.

6. The system according to claim 1, wherein the numeric portion of the code represents the number of monosaccharides attached to a branch of an N-linked glycan core structure.

7. The system according to claim 1, wherein the alpha portion represents the type of linkage and specific sugar molecule attached to a branch of an N-linked glycan core structure.

8. The system according to claim 1, wherein the code comprises six alpha-numeric characters respectively representing the six linkage sites on an N-linked glycan core structure.

9. The system according to claim 8, wherein the first four branches of the N-linked glycan core structure are represented by odd numbers if the branch is a complex type and high-mannose branches are represented by letters.

10. The system according to claim 9, wherein:
complex branches terminating as a GlcNAc, galactose or neuraminic acid residue are represented by the number 3, 5 or 7 respectively;
the mannose residues of hybrid and high-mannose N-linked glycans are represented by the letters A-F, with each letter A, B, C, D, E, and F respectively designated as an even number 2, 4, 6, 8, 10, and 12;
for each branch, the letter value corresponds to double the number of mannose residues attached to that branch;
the fifth and sixth characters are digits having a value of 3 if a bisecting GlcNAc and fucose residue are present, respectively; and
if a branch is not present, its corresponding number is 1

11. The system according to claim 1, wherein the code comprises seven alpha-numeric pairs.

12. The system according to claim 11, wherein the first through fifth alpha-numeric pairs respectively represent the five linkage sites on an N-linked glycan core structure, the sixth alpha-numeric pair represents a bisecting GlcNAc between the mannoses, and the seventh position corresponds to fucose molecules that can be attached to the core or peripheral GlcNAc residues.

13. The system according to claim 11, wherein the seventh alpha-numeric pair represents fucosylation on N-acetylglucosamine residues attached to the oligonucleotide.

14. The system according to claim 1, wherein the oligosaccharide is an N-glycan structure and is secreted glycoproteins from mammalian cell cultures.

15. The system according to claim 1, wherein the system further includes a difference operator defined to qualitatively differentiate between glycan structures.

16. A method for representing the structure of at least a portion of an oligosaccharide, comprising the steps of:
(a) selecting a base oligosaccharide structure;
(b) identifying a number of possible substitution points on the base structure selected in step (a) and assigning a position to each one;
(c) assigning a two-character code to a substitution point from step (b), where “character” means any unique identifier, the two-character code having a first character and a second character;
(d) assigning one or more unique identifiers for the first character of the two-character code and one or more unique identifiers for the second character of the two-character so that the first character and the second character together uniquely identify a residue on a specific substitution point identified in step (b);
(e) repeating step (d) for each substitution point so that each substitution point identified in step (b) has a set of two-character codes which identify the possible residues for that substitution point;
wherein the first character is a number corresponding to the number of residues attached at the substitution point branch represented by the two-character code; and the second character is a letter that serves as an index to a table containing additional information about the type of linkage and the type of linkage and the specific residue added.

17. The method of claim 16, further comprising reviewing the structure of an oligosaccharide structure containing the selected base oligosaccharide structure and optionally one or more residues on that base structure; and assigning the two-character codes to the residues on the oligosaccharide structure to match the two-character codes developed in steps (d) and (e) and recording them in the positions assigned in step (b).

18. The method of claim 16, wherein the base oligosaccharide structure of step (a) is an N-linked glycan structure.

19. The method according to claim 18, wherein the N-linked glycan structure is one of a complex, high-mannose and hybrid type.

20. The method according to claim 16, wherein the residues which are uniquely identified by the first and second characters in step (d) are selected from the group consisting of mannose, N-acetylglucosamine, galactose, fucose and sialic acid residues.

21. The method according to claim 18, wherein the first character of step (c) is a number.

22. The method according to claim 21, wherein the number represents the number of monosaccharides attached to a substitution point of an N-linked glycan core structure.

23. The method according to claim 21, wherein the second character of step (c) is a letter.

24. The method according to claim 23, wherein the letter represents the type of linkage and specific sugar molecule attached to a substitution point of an N-linked glycan core structure.

25. The method according to claim 19, wherein six substitution points are selected in step (b).

26. The method according to claim 25, wherein the first four substitution points of the N-linked glycan core structure are represented by odd numbers if the branch is a complex type and high-mannose branches are represented by letters.

27. The method according to claim 19, wherein seven substitution points are selected in step (b).

28. The method according to claim 27, wherein the first through fifth substitution points alpha-numeric pairs represent the five linkage sites on an N-linked glycan core structure, the sixth substitution point represents a bisecting GlcNAc between the mannoses, and the seventh substitution point corresponds to fucose molecules that can be attached to the core or peripheral GlcNAc residues.

29. The method according to claim 28, wherein the first character of step (c) is a number.

30. The method according to claim 29, wherein the second character of step (c) is a letter.

31. The method according to claim 18, wherein the oligosaccharide is an N-glycan structure and is secreted glycoproteins from mammalian cell cultures.

32. (canceled)
